(19) United States
(12) Patent Application Publication
(10) Pub. No.: US 2008/0134353 A1
(43) Pub. Date: Jun. 5, 2008

(54) Method Of Producing Haploid And Doubled Haploid Plant Embryos, And Embryos, Plants, Progeny, Cells, Tissues And Seeds Obtainable By Method

(76) Inventors: Robert Helene Ghislain Dirks, Oudenbosch (NL); Gerrit Cornelis Angenent, Wageningen (NL); Cecilia Lucia Clara Lelivelt, Oud-Beijerland (NL); Johannes Bernardus Maria Custers, Wageningen (NL)

Correspondence Address: FROMMER LAWRENCE & HAUG, 745 FIFTH AVENUE- 10TH FL., NEW YORK, NY, 10151, US

(21) Appl. No.: 11/947,915

(22) Filed: Nov. 30, 2007

Related U.S. Application Data
(63) Continuation-in-part of application No. PCT/EP2006/005238, filed on May 31, 2006.

(30) Foreign Application Priority Data
May 31, 2005 (EP) 05076264.0

(51) Int. Cl. A01H 1/02; A01H 5/00; C12N 5/04

(52) U.S. Cl. 800/260; 800/298; 435/419

(57) ABSTRACT

The invention relates to a method for producing haploid plant embryos, comprising providing microspores or pollen that comprise cell division inducing molecules; pollinating an embryo sac cell, in particular an egg cell, of the plant of which the haploid embryo is to be made with the microspores or pollen; allowing the microspores or pollen to discharge the cell division inducing molecules in or in the vicinity of the embryo sac cell, in particular the egg cell, to trigger division thereof to obtain a haploid plant embryo. When doubled haploid plant embryos are to be produced doubling of the chromosome number takes place at a certain stage after pollination, in particular during cell division or after obtaining the embryo. The invention further relates to the embryos thus obtained, plants regenerated therefrom and progeny thereof.

### REFERENCE TO RELATED APPLICATIONS

This application is a continuation-in-part of International Patent Application PCT/EP2006/005238 filed May 31, 2006 and published as WO 2006/128707 on Dec. 7, 2006, which claims priority from European Patent Application No. 05076264.0 filed May 31, 2005.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to a new method of producing haploid and doubled haploid plant embryos. The invention further relates to the plant embryos thus obtained and to plants regenerated therefrom, and to progeny, cells, tissues and seeds of these plants.

### BACKGROUND OF THE INVENTION

Since the discovery by Guha & Maheshwari in 1964 (Nature 204: 497) that plants can be regenerated from haploid spores, a lot of research has been done to obtain similar knowledge for other species (see e.g. “In vitro Haploid production in Higher plants” Vol. 1, 2, 3, 4, 5, Eds: S. Jain, S. Sopory and R. Veilleux (1996) Kluwer Academic Publishers).

In modern, contemporary plant breeding the use of doubled haploids (DHs) has become a very valuable tool in order to speed up the creation of genetically pure lines and also to evaluate and monitor difficult traits such as those that are encoded by multiple genes/alleles.

The production and the use of doubled haploids in breeding of crop plants is well known for many species (see e.g. Thomas W. et al. (2003), In: Doubled haploid production in crop plants. A Manual. Eds. M. Maluszynski, K. Kasha, B. Forster and I. Szarejko. Kluwer Academic Publishers, pp 337-349). In general, doubled haploids can be obtained from spores from the male organs. In this case the spores are named “microspores” and the in vitro cultures are called “microspore cultures”. Doubled haploids can also be obtained from the female organs, or “megaspores”. The corresponding in vitro culture is mostly named “gynogenesis”.

Typical microspore cultures are well established in Brassica since a long time (see e.g. Keller et al. (1984) In: K. Giles, S. Sen (eds.), Plant Cell Culture in Crop Improvement pp 169-183. Plenum Pub. Corp., New York). Typical gynogenesis cultures are known for sugarbeet (see e.g. Hosemans D. and Bossoutrot, Z. Pflanzenzüchtg. 91:74-77 (1983)). Also in cucumber, gynogenesis is a very well established technique (see EP 0 374 755).

A method that comes close to gynogenesis is based on the induction of embryogenesis of the egg cell by parthenogenesis making use of irradiated pollen. A well known example is published for melon and is currently routinely applied in several breeding companies (see Sauton A and R. Dumas de Vaulx, Agronomie 7: 141-148 (1987)). In parthenogenesis a new plant develops from an unfertilized egg. The success rate of this technique is low.

In spite of the wealth of available technologies and experiences in more than 30 years of research, the success of many techniques is limited to amenable genotypes. This means that the enormous benefits of the use of DHs cannot be utilized to its full extent. Apart from the variation in responsiveness of certain genotypes, several crop species such as tomato and cotton are still recalcitrant for induction of DHs.

It is therefore the object of the present invention to provide a new method for producing haploid or doubled haploid plant embryos.

In the research that led to the present invention it was surprisingly found that egg cells can be induced to undergo embryogenesis by pollinating them with pollen or microspores that comprise cell division inducing molecules. The egg cell is then triggered to form an embryo without fertilization. Because the pollen do not actually fertilize the egg cell no diploid zygote is formed and the resulting embryo remains haploid. At a certain stage after pollination during cell division spontaneous chromosome doubling can occur resulting in embryos that are in part or completely DHs. Chromosome doubling can also be induced, for example by known chemical means such as colchicine.

The invention thus relates to a method for producing haploid plant embryos, comprising the steps of:
a) providing microspores or pollen that comprise cell division inducing molecules;
b) pollinating an embryo sac cell, in particular an egg cell, of the plant of which the haploid embryo is to be made with the microspores or pollen;
c) allowing the microspores or pollen to discharge the cell division inducing molecules in or in the vicinity of the embryo sac cell, in particular the egg cell, to trigger division thereof to obtain a haploid plant embryo.

In an alternative embodiment, the invention relates to a method for producing doubled haploid plant embryos, comprising the steps of:
a) providing microspores or pollen that comprise cell division inducing molecules;
b) pollinating an embryo sac cell, in particular an egg cell, of the plant of which the doubled haploid embryo is to be made with the microspores or pollen;
c) allowing the microspores or pollen to discharge the cell division inducing molecules in or in the vicinity of the embryo sac cell, in particular the egg cell, to trigger division thereof to obtain a plant embryo, wherein doubling of the chromosome number takes place at a certain stage after pollination, in particular during cell division or after obtaining the embryo.

The invention thus relates to the use of pollen or microspores as a vehicle to trigger cell division in the embryo sac cell or egg cell.

In a first embodiment, the cell division inducing molecules are transiently expressed in the pollen, for example from a nucleic acid that is present on a plasmid. The cell division inducing molecules, which can be either nucleic acid or protein, are produced in the pollen or microspores by constitutive expression from the plasmid. The thus produced cell division inducing molecules are discharged into the egg cell after pollination.

In a second embodiment, the cell division inducing molecules are expressed from a nucleic acid that is stably incorporated in the pollen genome. The cell division inducing molecules, which can be either nucleic acid or protein, are produced in the pollen or microspores by constitutive expression. The thus produced cell division inducing molecules are discharged in the embryo sac or egg cell after pollination.

In a third embodiment, the cell division inducing molecule is produced in the embryo sac cell or egg cell by expression from a nucleic acid that is under the control of an embryo sac cell- or egg cell-specific promoter, which nucleic acid is brought into the embryo sac cell or egg cell by means of pollination with pollen or microspores comprising the nucleic acid. In the embryo sac cell or egg cell the tissue-specific promoter is switched on thus leading to the production of the cell division inducing molecules.

In a fourth embodiment, the pollen or microspores are not directly transformed but are formed on a transgenic plant that carries the nucleic acid encoding the cell division inducing molecule. Such transgenic pollen or microspores may be under the control of a constitutive promoter or a pollen- or microspore-specific promoter or an embryo sac cell- or egg cell-specific promoter. In the first case, it is preferred that the transcript is not detrimental to the plant. In the second case, the transgene is only expressed in the pollen or microspores and the cell division inducing molecules are only produced in the pollen or microspores. In the third case, the transgene is expressed when the nucleic acid enters the egg cell or embryo sac cell after pollination.

In all these embodiments, actual fertilization of the egg cell by the pollen must be avoided. This can be achieved by means of irradiation or by using different species for the pollen and egg cells. Irradiation is in particular suitable for embodiments in which protein is produced in the pollen as protein will not or virtually not be damaged by the irradiation. In cases where nucleic acid is to be transferred from the pollen or microspores to the embryo sac or egg cell, the use of another species as the egg cell donor is preferred.

According to a particular embodiment of the invention, the microspores or pollen that comprise cell division inducing molecules are obtainable by transformation with a nucleic acid. The transformation can be performed in any suitable way, such as by means of Agrobacterium tumefaciens or by means of particle bombardment (biolistics).

These transformation techniques are well known. Transformation of plant cells by means of Agrobacterium tumefaciens is well established and for example reviewed in De la Riva et al., EJB Vol. 1(3) (1998), and Bent, Plant Physiol. 124:1540-1547 (2000).

Recently, it was discovered that genetic transformation of plants is not solely restricted to Agrobacterium, but that other bacteria too have the capacity to transform plants (Broothaerts et al., Nature 433, 629-633 (2005), incorporated herein by reference). These plant-associated symbiotic bacteria were made competent for gene transfer by acquisition of both a disarmed Ti plasmid and a suitable binary vector. Such transformation systems are also suitable for use in the invention.

Biolistic transformation is also well known to the person skilled in the art and tools for such applications are commercial available since several years (Ralph Bock, In: QiagenNews, Issue No. 5, 1997).

Suitable techniques for use in the invention are for example also described by Barinova et al. (J Exp Bot. 53(371): 1119-29 (2002)), in which delivery of DNA at the level of microspores and transient expression thereof in Antirrhinum majus is shown, or by Ramaiah et al. (Current Science 73:674-682 (1997)) for alfalfa (Medicago sativa L.).

Methodology for microspore or pollen transformation with biolistic bombardment in tobacco can be found in Baubak Bajoghli, (Matrikel number: 9802743, University of Vienna, Experimentelle Genetic III. Plant Biotechnology by Alisher Touraev, July 2001). Van der Leede-Plegt, et al., Transgenic Research 4(2):77-86 (1995) describe direct delivery of DNA into pollen of tobacco (Nicotiana glutinosa) by means of microprojectile bombardment. These and other techniques can be used for the transformation of pollen or microspores for use in the invention.

In a particular embodiment, the pollen and microspores thus comprise the cell division inducing molecules by virtue of the presence of a nucleic acid. The nucleic acid that is introduced can be the cell division inducing molecule itself, or can encode the cell division inducing molecule. In the latter case the inducing molecule is a protein or peptide. In the first case the inducing molecule is a nucleic acid. The nucleic acid can be inducible in itself or it can block other nucleic acids from being expressed. For example, the nucleic acid can be or code for a RNAi against members of the Kip-related protein family or retinoblastoma (see e.g. Park J et al., Plant Journal 42:153-163 (2005)). Retinoblastoma protein regulates cell proliferation, differentiation, and endoreduplication in plants.

Alternatively, the nucleic acid can code for a precursor of the cell division inducing molecule or enzyme that produces the cell division inducing molecule. When the nucleic acid encodes an enzyme this may be an enzyme that is or directly produces the inducing molecule or be an enzyme that is part of a pathway that eventually leads to the inducing molecule. “Cell division inducing molecules” as used herein are intended to encompass all molecules that directly or indirectly trigger cell division.

The invention is based on the principle that cell division inducing molecules are delivered to the embryo sac or egg cell by means of transformed pollen or microspores.

Gene constructs or molecules that are capable of switching on cell division are in itself known and can be used in the new method of the invention.

Examples of genes that can be used according to the invention are described in Stone et al. (PNAS 98, 11806-11811 (2001)) disclosing that somatic cells (vegetative cells) that are programmed to continue vegetative growth are amenable to conversion to embryonic growth by transforming them with (a) gene(s) that encode transcription factors. Another example is Baby Boom (Boutilier et al., The Plant Cell 14, 1737-1749 (2002)) or leafy cotyledon (Stone S et al., PNAS 25:11806-11811 (2001)). These and other genes can be used to encode the cell division inducing molecules of the invention.

Zuo et al. (The Plant Journal 30:1-12 (2002)) describes that somatic embryogenesis was found to be induced without plant hormones in all tissues of Arabidopsis by over-expressing the so-called Wuschel gene. This technology is also described in US2003/0082813. The difference with the present invention is that in US2003/0082813 a plant cell is stably, not transiently, transformed with a Wuschel DNA sequence. This stably introduced Wuschel gene is then overexpressed in the tissue in which it is stably incorporated and in this tissue cell division and embryo formation is induced.

According to the invention, the nucleic acid that encodes the cell division inducing molecule, which may be the expression product of the Wuschel gene, is not stably incorporated in the genome of the cell that should undergo cell division. The nucleic acid encoding the cell division inducing molecule is either transiently expressed in the dividing cell because it is under the control of an egg cell-specific promoter or expressed in the pollen after which the encoded cell division inducing molecules are discharged in the cell (egg cell or embryo sac cell) that is to be triggered to start cell division. The pollen or microspore are thus a vehicle to introduce the molecule itself (the Wuschel gene expression product, not the coding sequence) into the cell to be triggered to start cell division.

The application of egg cell inducing genes is not restricted to ectopic and transient expression of the genes like the ones mentioned above but similar results can also be obtained by using genes that encode enzymes that can produce hormones such as iaaM and iaaH (see Thomashow et al., (1986) Science 231, 616-618) and genes that encode cell cycle proteins.

In addition, combinations of genes can be used for further optimizing induction of division and embryogenesis of the egg cell. Transient expression of cell cycle genes can also provoke divisions of the egg cell (for a review of cell cycle genes see Murray A (Cell 116:221-234 (2004)). In particular transient expression of cyclin E and D alone or in combination can be used to trigger egg cell divisions.

According to the invention, the nucleic acid is either expressed in the microspores or pollen, either transiently or after stable integration in the genome, or transiently expressed from an egg cell-specific promoter in the egg cell. This way constitutive expression in the resulting embryo is avoided.

Transient expression can occur in a tissue- or cell-specific manner. In an alternative embodiment transient expression is obtained by putting the nucleic acid under regulation of a tissue specific or inducible promoter. The tissue specific promoter is suitably a pollen- or microspore-specific promoter. Pollen-specific promoters are well known and transient expression has been shown both in monocot and dicot species. Examples of these types of promoters are for example described in Twell, D et al., Development 109(3):705-713 (1990); Hamilton, D et al., Plant Mol. Biol. 18:211-218 (1992).

Thus, according to the invention, by using conventional ways of transforming microspores (such as by Agrobacterium transformation or biolistic transformation) or pollen grains, that leads to transient expression of genes like Baby Boom, Wuschel, leafy cotyledon, cyclines, cyclin dependent kinases (CDK), E2F (member of a transcription factor family in higher eukaryotes; Zheng, N et al. Genes Dev. 13:666 (1999)), DP (Magyar Z et al., FEBS Lett. 486(1):79-87 (2000)) etc. it is possible to induce divisions of the egg cell when the female recipient is pollinated with the transformed microspores or pollen grains.

Preferably, the generative nucleus of the pollen or microspores is inactivated or destroyed. This way fertilization of the egg cell is certainly avoided. Inactivation or destruction of the generative nucleus is preferably performed before transformation of the pollen or microspores in order not to unnecessary damage the inducing molecules. Inactivation or destruction is suitably effected by means of irradiation.

Irradiation of pollen nuclei is a well known method to degrade the generative nucleus, but dependent on the dose of irradiation it does not obstruct the pollen tube to be formed and discharge into the egg cell. Grant et al. (New Zealand Journal of Botany 18, 339-341 (1980)) describes this technique.

Irradiated and subsequently transformed pollen/microspore cells are subsequently transferred onto the pistils of plants from the same species or a species in which pollen discharge of the said pollen/microspore cells can occur. An example of heterologous pollination is the use of a species belonging to the Solanaceae family as a pollen donor and tomato as an acceptor. Other examples are described in de Martinis, D et al. Planta 214(5):806-812 (2002) and Dore C et al., Plant Cell Reports 15:758-761 (1996). In general, species that are suitable for heterologous pollination belong to the same family.

Dependent on the species, seeds can be harvested that have originated from division from the egg cell or ovary. Alternatively, ovule culture may be necessary to rescue the developing embryo.

Thus, basically the inactivated pollen grain carries in a transient way signal molecules that are capable of inducing egg cell division and embryogenesis. Because of the transient nature of the molecules, the egg cell DNA is not stably transformed.

The invention thus relates in a particular embodiment to the utilization of cell division molecules (proteins, DNA, RNA) that are transiently present and expressed in microspores or pollen grains that were inactivated by irradiation in order to inactivate the generative nucleus, and whereby the said cell division molecules exert their action when discharged by the pollen tube in or in the vicinity of the egg cell.

In another embodiment, the expression of egg cell inducing molecules is transient even though the donor plant of the pollen or microspores is stably transformed with genes encoding cell division inducing molecules. In this specific embodiment, the plant donating the pollen or microspores is stably transformed so that its genomic DNA carries genes or gene constructs that encode cell division molecules, preferably under the control of an embryo sac cell- or egg cell-specific promoter. The genes or gene constructs are then expressed when released in an embryo sac cell, in particular in an egg cell, or its vicinity. The resulting cell division inducing molecules trigger cell division.

Prior to pollination, the pollen or microspores are irradiated to inactivate the generative nucleus. Alternatively, pollen or microspores are transferred onto the pistil of another species in which pollen discharge of the said pollen/microspore cells can occur. The advantage of this method is that the nucleic acids encoding the cell division inducing molecules are carried in the generative nucleus and eventually end up in the sperm cells. Preferably, multiple copies of the cell division inducing molecules are present in the donor plant and by consequence in the sperm cells. To avoid interference of the presence of the gene constructs with the development of the pollen or microspores, inducible or specific promoters, preferably embryo sac cell or egg cell specific promoters, are used that enable the expression of the genes or gene constructs only when they are transferred into the embryo sac cell, in particular the egg cell.

The invention further relates to haploid embryos and doubled haploid embryos, obtainable by means of the method of the invention, as well as to plants regenerated from such haploid embryos or doubled haploid embryos, progeny of such plants, and to seeds, cells, tissues, microspores and egg cell from such a plant or progeny thereof.

In the present application the word egg cell is sometimes used alone for the purpose of legibility but is then intended to be read as “embryo sac cell, in particular egg cell”.

The present invention will be further elucidated in the Examples that follow and that are intended for illustration purposes only and are not to be construed as limiting this invention in any way.

### BRIEF DESCRIPTION OF THE DRAWINGS

FIG. 1 is a photograph of three inflorescences of the Arabidopsis p35S:AP2mut as placed in the middle of a petri dish.

FIG. 2 is a photograph of a GUS positive pollen obtained from gold particle bombardment of the three inflorescences shown in FIG. 1.

FIGS. 3A-3D are photographs of GUS positive pollen obtained from gold particle bombardment where the petri dish was placed on level 3 in the particle gun.

FIG. 4 is a photograph showing 15 different flower stages of tomato.

FIG. 5 is a photograph showing a representative example of GUS positive pollen tubes.

FIG. 6 is a vector diagram depicting pExo70::GFP:GUS.

FIG. 7 is a vector diagram depicting pES4::ES4:GFP.

### EXAMPLES

### Example 1

### Transformation of Arabidopsis Pollen by Means of Particle Bombardment

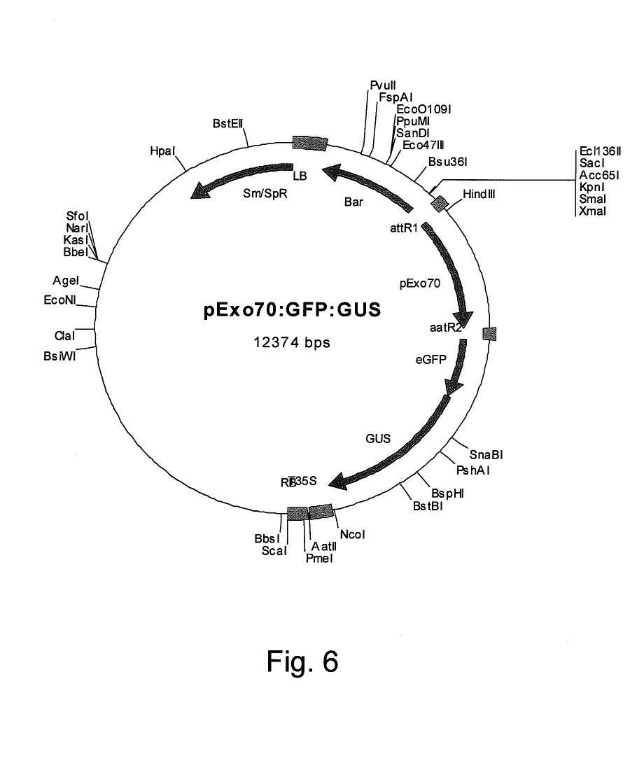

The DNA plasmids pCAMBIA 1301 and pExo70::GFP:GUS were used to coat 1 μm gold particles. pCAMBIA 1301 is a binary vector, which contains GUS regulated by an 800 nucleotide CaMV 35S promoter (Roberts et al., pCAMBIA Vector release manual version 3.05 (1998)). pExo70::GFP:GUS contains -glucuronidase (GUS) and green fluorescent protein (GFP) (FIG. 6).

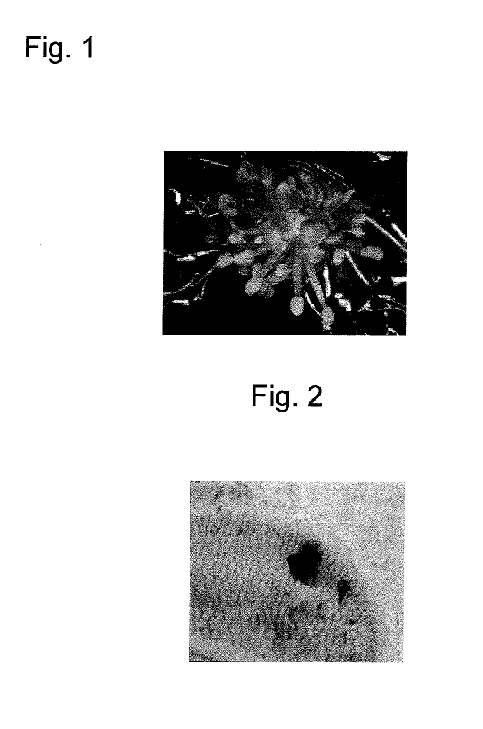

Three inflorescences of the Arabidopsis p35S:AP2mut were placed in the middle of a petri dish (FIG. 1). The petri dish was placed in the particle gun and three shots of the coated gold particles were fired. Two days after bombardment expression was studied. FIG. 2 shows a GUS positive pollen thus obtained.

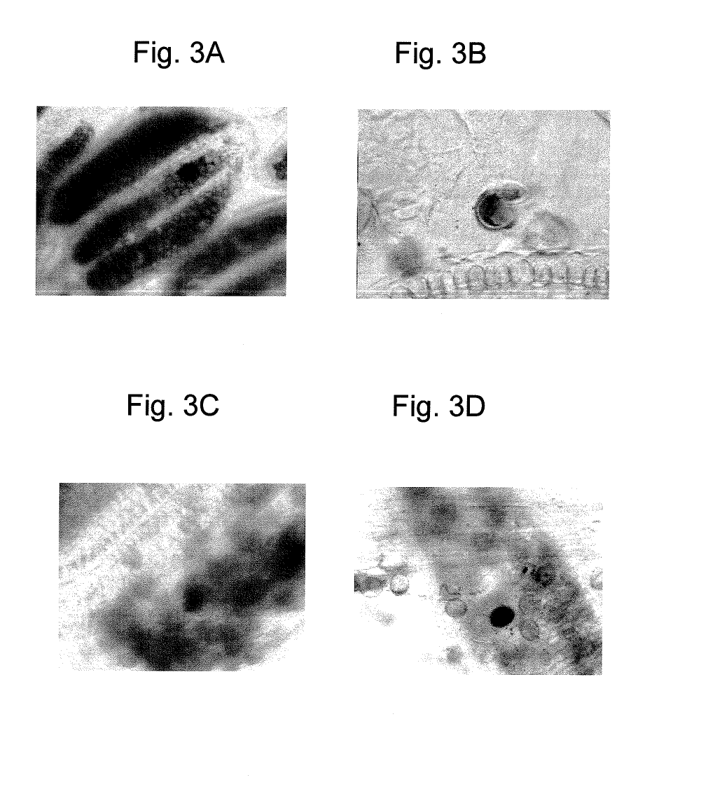

The same experiment was repeated with the petri dish on level 3 in the particle gun. Two shots were fired at a pressure of 2200 psi. FIGS. 3A-D show representative results of this experiment.

It follows from this experiment that particle bombardment can be used to transform pollen.

### Example 2

### In Vitro Pollen Germination After Particle Bombardment

Mature pollen is quiescent. After deposition of the pollen grain on the stigma of a female plant the process of pollen germination begins with rehydration through water transfer from the stigma. In the present example the pollen are germinated in vitro after particle bombardment.

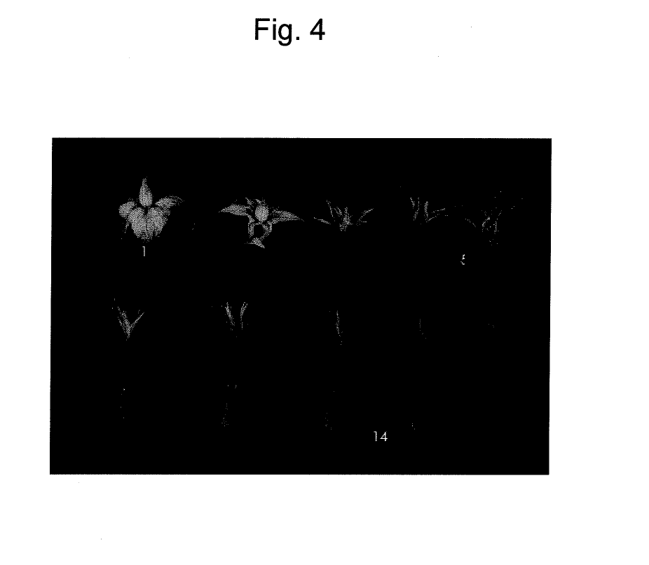

FIG. 4 shows 15 different flower stages of tomato. Pollen of stage 1, 5 and 14 was used in this experiment. Pollen of stage 1 is completely mature and pollen of stage 5 is also mature. Stage 14 is the late-uni/early binucleate phase.

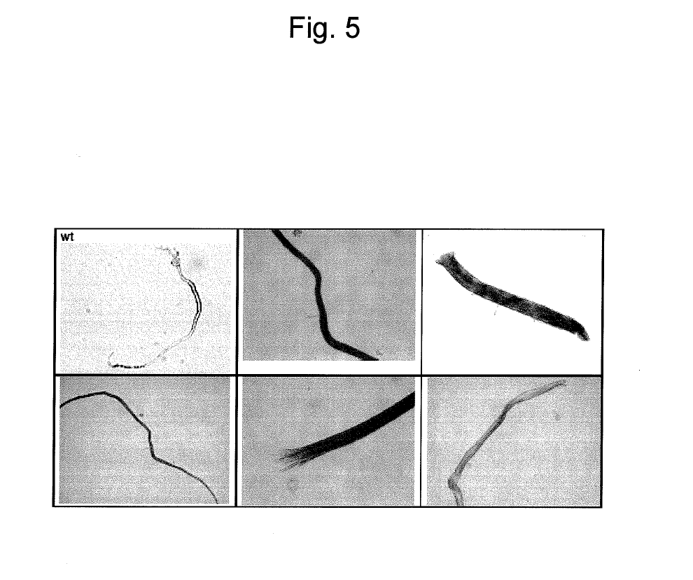

Pollen of flowers in stage 1, 5 and 14 was isolated in 200 μl NLN13 medium (NLN medium (Lichter R., Z Pflanzenzuecht 105:427-437 (1982)) supplemented with 13% sucrose). The 200 μl are spotted on a genescreen membrane and dried for 5 min. Then the membrane is placed on a ½MS agar plate and bombarded at 2200 psi with 1 μm gold particles coated with Exo70::GFP:GUS. After bombardment, the membranes are placed in a 6 well titre plate. Pollen of stage 1 and 5 is incubated in 1.5 ml germination medium A (Clarke) (20 mM MES, 0.07% Ca(NO₃)₂.H₂O, 0.02% MgSO₄.7H₂O, 0.01% KNO₃, 0.01% H₃BO₃, 2% sucrose and 15% PEG4000). Microspores of stage 14 are incubated in NLN13 medium. After three hours of incubation, 1.5 ml 2×GUS staining buffer was added and the samples were placed at 37° C. overnight. FIG. 5 shows a representative example of GUS positive pollen tubes. Clearly, pollen is still capable of forming a tube after transformation.

### Example 3

### Preparation of Pollen Carrying a Cell Division Stimulating Factor for Inducing Cell Division of the Egg Cell After Pollination with These Pollen

Examples 1 and 2 demonstrate that pollen can be transformed in a model system with GUS. Here it is described how tomato pollen is transformed with the cell division inducing molecule BabyBoom (BBM)(Boutilier et al., 2002, supra) after irradiation of the pollen.

Pollen from a stably transformed plant carrying the CaMV 35S promoter::GFP construct were used. This construct is used as a visible non-destructive marker to discriminate between embryos and endosperm derived from a sexual event and embryos derived via the method of the invention. The CaMV 35S promoter is active in embryos and endosperm, but not in ovules and therefore only mark the sexually derived embryos. The plant that was used as pollen donor was homozygous for this CaMV 35S promoter::GFP construct.

The pollen were irradiated and the irradiation dose was selected in such a way that still a few pollen were able to fertilize the egg cell and induce normal zygotic embryo formation upon pollination. This also stimulated the outgrowth of the ovary to a fruit, which contained less than 10% of the normal number of seeds indicating that the sexual reproduction process was not completely abolished, but was severely affected.

Transformation is performed with particle bombardment as described in Example 2 with the BBM gene driven by the promoter sequence of the Arabidopsis Exo70 gene (Atg28640). The construct contains the EXO70 promoter sequence (pExo70) fused to BBM.

### Example 4

### Pollination with Transformed Pollen and Embryogenesis

Tomato flowers were emasculated and pollinated with the transformed pollen obtained in Example 3. After pollination, the ovaries expanded and formed fruit-like bodies. The young fruit-like structures were kept on the plants for 2-4 weeks. Plants were grown under climatized conditions (22° C. day, 18° C. night). Fruits were harvested and imaged for GFP expression (CaMV-35S::GFP) to eliminate the sexually derived embryos.

A part of the ovules initiated a parthenogenic development according to the invention and produced embryo-like structures without showing GFP fluorescence. The rescued embryos were further incubated on medium as described by Neal, C A and Topoleski, L D (J. Amer. Soc. Hort. Sci. 108(3):434-438 (1983). Between 25%-50% of the immature embryos were able to regenerate into viable plantlets. None of the plantlets were transgenic, demonstrating the maternal origin of the embryos.

In addition, the origin of the embryos was also checked using male-specific molecular markers (Vos P. et al., Nucleic Acids Research, 23:4407-4414 (1995)).

In order to discriminate between haploid and diploid embryos, measurements of DNA-ploidy level were performed by flow cytometry according to the method described in De Laat, A et al., Plant Breeding 99:303-307 (1987). The majority of the obtained plantlets appeared to be doubled haploid.

### Example 5

### Expression Driven from an Egg Cell Specific Promoter

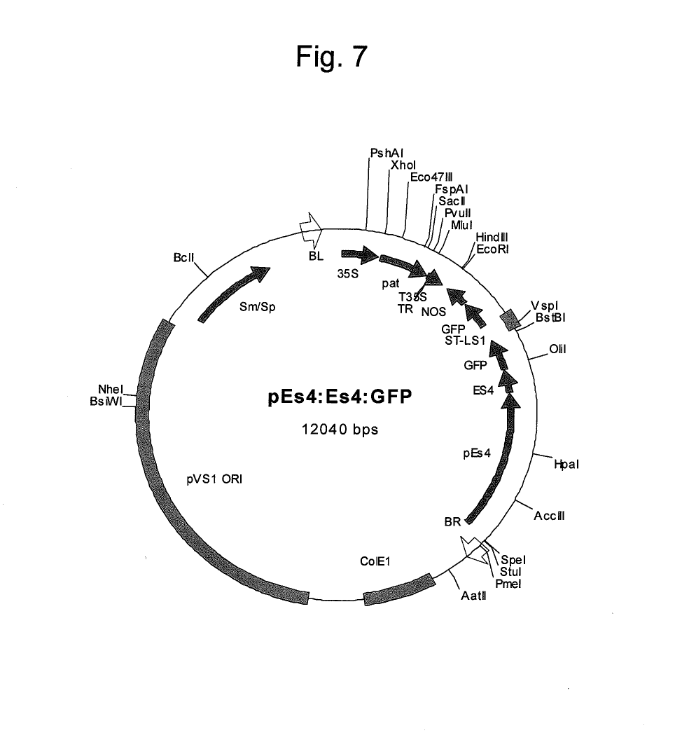

Pollen from Arabidopsis was transformed as described in Example 1 with the pES4::ES4:GFP construct (see FIG. 7) comprising the reporter gene GFP under the control of the pES4 promoter from maize (Cordts, S et al., The Plant J 25:103-114 (2001)). No fluorescence was observed in the transformed pollen.

After pollination of an Arabidopsis plant with the transformed pollen fluorescence was specifically detectable in the egg cell. This experiment demonstrates that the pES4 promoter is active in the egg cell and can be used to switch on the expression of cell division inducing molecules.

1. A method for producing haploid plant embryos, comprising the steps of:
a) providing microspores or pollen that comprise cell division inducing molecules;
b) pollinating an embryo sac cell, in particular an egg cell, of the plant of which the haploid embryo is to be made with the microspores or pollen;
c) allowing the microspores or pollen to discharge the cell division inducing molecules in or in the vicinity of the embryo sac cell, in particular the egg cell, to trigger division thereof to obtain a haploid plant embryo.

2. A method for producing doubled haploid plant embryos, comprising the method of claim 1, wherein doubling of the chromosome number takes place at a certain stage after pollination, in particular during cell division or after obtaining the embryo.

3. The method of claim 2, wherein doubling of the chromosome number occurs spontaneously.

4. The method of claim 2, wherein doubling of the chromosome number is effected by means of chemical treatment, in particular by means of colchicine.

5. The method of claim 1, wherein the microspores or pollen that comprise cell division inducing molecules are obtainable by transformation of microspores or pollen with a nucleic acid or are obtainable from plants that are transformed with a nucleic acid encoding a cell division inducing molecule.

6. The method of claim 5, wherein the transformation is performed by means of Agrobacterium tumefaciens or biolistics.

7. The method of claim 5, wherein the nucleic acid is the cell division inducing molecule or encodes the cell division inducing molecule, precursor of the cell division inducing molecule or enzyme that produces the cell division inducing molecule.

8. The method of claim 5, wherein the nucleic acid which is the cell division inducing molecule is a RNAi that blocks expression of genes that inhibit or arrest cell division.

9. The method of claim 8, wherein the gene that inhibits or arrests cell division is retinoblastoma (Rb) or a member of the Kip-related protein (KRP) family.

10. The method of claim 5, wherein the cell division inducing molecule is selected from the group consisting of Baby Boom, Leafy cotyledon, WUSCHEL, cyclines, cyclin dependent kinases (CDK), E2F, DP.

11. The method of claim 5, wherein the nucleic acid is transiently expressed in the microsporesor pollen.

12. The method of claim 11, wherein transient expression is obtained by putting the nucleic acid under regulation of a tissue specific or inducible promoter.

13. The method of claim 12, wherein the tissue specific promoter is a pollen- or microspore-specific promoter.

14. The method of claim 5, wherein the nucleic acid is stably incorporated in the microspores or pollen but transiently expressed in the embryo sac cell or egg cell.

15. The method of claim 14, wherein transient expression is obtained by putting the nucleic acid under regulation of a tissue specific or inducible promoter that is not active in the pollen or microspores.

16. The method of claim 12, wherein the tissue specific promoter is an embryo sac or egg cellspecific promoter.

17. The method of claim 15, wherein the tissue specific promoter is an embryo sac or egg cellspecific promoter.

18. The method of claim 1, wherein the microspores or pollen in which the nucleic acid encoding the cell division inducing molecule is stably incorporated or transiently expressed are from a donor plant that belongs to another species than the acceptor plant that donates the embryo sac cell or egg cell.

19. The method of claim 1, wherein the generative nucleus of the pollen or microspores is inactivated or destroyed.

20. The method of claim 19, wherein the inactivation or destruction of the generative nucleus is performed before transformation of the pollen or microspores.

21. The method as claimed in claim 20, wherein the inactivation or destruction of the generative nucleus is effected by means of irradiation.

22. A haploid plant embryo, obtainable by means of the method of claim 1.

23. A doubled haploid plant embryo, obtainable by means of the method of claim 2.

24. Plants regenerated from plant embryos as claimed in claim 22.

25. Plants regenerated from plant embryos as claimed in claim 23.

26. Progeny of plants as claimed in claim 24.

27. Progeny of plants as claimed in claim 25.

28. Seeds of a plant as claimed in claim 24.

29. Seeds of a plant as claimed in claim 25.

30. Seeds of a plant as claimed in claim 26.

31. Seeds of a plant as claimed in claim 27.

32. Cells from a plant as claimed in claim 24.

33. Cells from a plant as claimed in claim 25.

33. Cells from a plant as claimed in claim 26.

34. Cells from a plant as claimed in claim 27.

35. Cells as claimed in claim 32, which cells are selected from pollen, microspores, embryo sac cells and egg cells.

36. Cells as claimed in claim 33, which cells are selected from pollen, microspores, embryo sac cells and egg cells.

37. Cells as claimed in claim 34, which cells are selected from pollen, microspores, embryo sac cells and egg cells.

38. Cells as claimed in claim 35, which cells are selected from pollen, microspores, embryo sac cells and egg cells.

39. Tissue from a plant as claimed in claim 24.

40. Tissue from a plant as claimed in claim 25.

41. Tissue from a plant as claimed in claim 26.

42. Tissue from a plant as claimed in claim 27.

43. Tissue as claimed in claim 39, which tissue is embryo sac tissue.

44. Tissue as claimed in claim 40, which tissue is embryo sac tissue.

45. Tissue as claimed in claim 41, which tissue is embryo sac tissue.

46. Tissue as claimed in claim 42, which tissue is embryo sac tissue.
